(19) United States
(12) Patent Application Publication
(10) Pub. No.: US 2017/0192021 A1
(43) Pub. Date: Jul. 6, 2017

(54) OPTICAL BILIRUBIN SENSOR AND ASSAY

(71) Applicant: Magellan Diagnostics, Inc., North Billerica, MA (US)

(72) Inventors: Norman F. Sheppard, JR., New Ipswich, NH (US); Gary Conrad Jensen, Cambridge, MA (US); Roland Lawrence Guilmet, Lowell, MA (US); Peter Michael Magnin, Merrimack, NH (US)

(21) Appl. No.: 15/394,452

(22) Filed: Dec. 29, 2016

Related U.S. Application Data
(60) Provisional application No. 62/273,220, filed on Dec. 30, 2015.

(51) Int. Cl. G01N 33/72; G01N 33/52; G01N 21/31

(52) U.S. Cl. CPC G01N 33/728; G01N 21/31; G01N 33/526

(57) ABSTRACT

A sensor and method for measuring bilirubin in a liquid sample are disclosed. The sensor comprises a substrate comprising a reservoir disposed within the substrate, the reservoir having a top surface and a bottom surface; a filter; at least one transparent portion, the transparent portion forming at least a part of the bottom surface of the reservoir, and a reflector comprising at least a portion of the reservoir. The method for measuring bilirubin in a liquid sample comprises inserting a sensor into an analyzer; introducing the liquid sample to the sensor; filtering the liquid sample such that the sample flows into a reservoir in the sensor; illuminating the liquid sample in the sensor using a light source in the analyzer; measuring a reflectance of the liquid sample at one or more wavelengths using a detector in the analyzer; and computing a measurement of bilirubin using the measured reflectances.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the benefit of U.S. Provisional Application No. 62/273,220 filed on Dec. 30, 2015.

### BACKGROUND

Field

This disclosure relates to a sensor and method for analyzing bilirubin in a sample.

Description

The detection of abnormal serum bilirubin levels can be used for the detection of a variety of health issues ranging from jaundice in children to liver disease in adults. For newborn infants, raised serum bilirubin levels (hyperbilirubinemia) affect between 50 percent of term and 80 percent of preterm infants, leading to jaundice within their first week of life. Akobeng, A. “Neonatal Jaundice,” Am. Fam Physician 2005, 71(5):947-948. In addition to producing jaundice, unconjugated bilirubin can penetrate the blood-brain barrier of newborn infants. Newborn infants with high bilirubin levels in the brain may develop acute, chronic or subtle bilirubin encephalopathy. These disorders can produce long-term debilitating effects such as hearing loss, movement disorders, auditory dysfunction, and oculomotor impairments, or in severe cases, seizures or death. In adults, raised bilirubin serum levels may be a symptom of a number of serious illnesses such as hepatitis, cirrhosis, fatty liver disease, or liver cancer. For these reasons, a quick and accurate assay for measuring serum bilirubin has significant implications for public health.

When sampling blood bilirubin, an optical assay may be desirable as a method for an accurate and quick testing. Some exemplary techniques for facilitating the binding and measurement of serum bilirubin levels are found in U.S. Pat. No. 3,569,721 “Measuring Bilirubin in Blood Using Light at Two Wavelengths,” U.S. Pat. No. 4,069,017 “Colorimetric Assay for Bilirubin,” U.S. Pat. No. 4,412,005 “Determination of Total Bilirubin,” and U.S. Pat. No. 4,788,153 “Method for the Determination of Bilirubin and an Element Useful Therein,” the entirety of which are incorporated herein by reference. U.S. Application Nos. 62/096,178 and Ser. No. 14/978,292, entitled “Combination Optical Hemoglobin and Electrochemical Lead Assay,” are also incorporated herein by reference.

### SUMMARY

In one aspect, a sensor for measuring bilirubin in a liquid sample comprises a filter; a reservoir having a top surface and a bottom surface; at least one transparent portion, the transparent portion forming at least a part of the bottom surface of the reservoir; and wherein a portion of the top surface comprises a reflector, is disclosed.

In some embodiments, the substrate further comprises a base layer forming the bottom surface of the reservoir, wherein the at least one transparent portion forms at least a portion of the base layer; a reflective layer having a void extending through a thickness of the reflective layer and wherein at least a portion of the reflective layer comprises a reflector; a filter layer wherein at least a portion of a bottom surface of the filter layer comprises a portion of the top surface of the reservoir; a spacer layer having a void extending through a thickness of the layer and wherein a portion of a bottom surface of the spacer layer may comprise a portion of the top surface of the reservoir; a lid, the lid having a void extending through a thickness of the lid, the lid having a bottom surface, and wherein at least a portion of the bottom surface of the lid forms at least a portion of the top surface of the spacer layer; and wherein the reflective layer is disposed on the base layer, the filter layer and spacer layer are disposed on the reflective layer, and the lid is disposed on the spacer layer.

In some aspects descried herein, a sensor for measuring bilirubin in a liquid sample comprises an optically transparent portion; a one or more electrodes formed on the base layer; a reflective layer, the reflective layer comprising: at least a portion of a reservoir, the reservoir configured to receive a sample to be analyzed.

In some embodiments, the sensor further comprises a filter layer disposed between at least a portion of a bottom surface of a lid and/or spacer layer and a top surface of a reflective layer.

In some embodiments, the sensor further comprises a spacer layer having a void extending through a thickness of the layer and wherein a portion of a bottom surface of the spacer layer may comprise a portion of the top surface of the reservoir;

In some embodiments, the sensor further comprises a spacer layer between the filter layer and the lid, the spacer layer having a void formed therein, the void extending through a thickness of the layer and wherein the size, shape, or thickness of the spacer layer determine the size, shape, or thickness of at least a portion of the filter layer.

In some embodiments, the sensor further comprises a lid, the lid having a void extending through a thickness of the lid, the lid having a bottom surface, and wherein at least a portion of the bottom surface of the lid forms at least a portion of the top surface of the spacer layer.

In some embodiments the filter inhibits the passage of erythrocytes, white blood cells, and/or platelets.

In some embodiments, at least a portion of the filter may be compressed by the lid and/or spacer layer.

In some embodiments a bilirubin binding material has been sputtered, printed, sprayed, air brushed, or otherwise deposited on at least a portion of the reflective layer.

In some embodiments, the bilirubin binding material is a cationic copolymer and gelatin.

In some embodiments, the reflector material comprises barium sulfate.

In some embodiments, the sensor comprises at least one electrode disposed on a bottom surface of the reservoir and at least one electrical contact disposed on the substrate, and wherein the at least one electrode is in electrical communication with the at least one electrical contact.

In some embodiments, the sensor comprises at least one electrode disposed on a bottom surface of the reservoir and at least one electrical contact disposed on the base layer, and wherein the at least one electrode is in electrical communication with the at least one electrical contact.

In another aspect described herein, a method for measuring bilirubin in a sample comprises inserting a sensor into an analyzer, the sensor comprising a filter and the analyzer comprising a light source and a detector; introducing the liquid sample to a sensor; filtering the sample using the filter; illuminating the liquid sample through a portion of the sensor using the light source; measuring a reflectance of the liquid sample at one or more wavelengths using a detector in the analyzer; and determining an amount of bilirubin based on the measured reflectances.

In some embodiments, the sample is whole blood and the filter inhibits the passage of erythrocytes.

In some embodiments, the sensor comprises a reflective layer, and reflectance is measured by measuring light reflected off the reflective layer.

In some embodiments, the method further comprises taking a reference measurement in the sensor when no sample is present in the sensor.

In some embodiments a reflectance is computed by comparing an intensity measured at the detector to a reference intensity.

In some embodiments the reference intensity is obtained by inserting a reference sensor into the analyzer, illuminating reference sensor, and measuring an intensity of light received at the detector.

In some embodiments, internally reflected stray light is measured by detecting at the detector the intensity of light reflected off a light absorbing surface as the sensor is inserted into or withdrawn from the analyzer, the method further comprising subtracting the measured internally reflected stray light from the reference intensity and the measured intensity of the sample to obtain a result which adjusts for internally reflected stray light.

In some embodiments, determining the bilirubin concentration comprises: subtracting the secondary wavelength absorbance value from the adjusted target wavelength absorbance value to obtain a result that is corrected for hemoglobin and plasma interference, the method further comprising comparing the resulting value to a bilirubin calibration curve to determine the bilirubin concentration.

In some embodiments, the target wavelength is approximately 480 nm.

In some embodiments, the secondary wavelength is approximately 525 nm.

### BRIEF DESCRIPTION OF THE DRAWINGS

FIG. 1 depicts a perspective view of an embodiment of an optical bilirubin sensor.

FIG. 2 depicts an embodiment of an analyzer configured to receive and analyze a sample in an optical bilirubin sensor.

FIG. 3A depicts an exploded, simplified (not to scale) longitudinal cross-sectioned view of an embodiment of an assembled sensor taken along the lines A-A′ of FIG. 1.

FIG. 3B depicts the sensor of FIG. 2A in an assembled state.

FIG. 4A is a cross-sectional view of the sensor of FIG. 1 taken along the line A-A′ having a fluid sample applied thereto and illuminated by light from an embodiment of an optical system.

FIG. 4B is a cross-sectional view of the sensor of FIG. 1 taken along the line A-A′ having a fluid sample applied thereto and illuminated by light from an embodiment of an optical system.

FIG. 5 is a depiction of the operation of the optical system in the analyzer used in FIG. 5A to measure the intensity of reflected light from an empty or filled reservoir.

FIG. 6 is a depiction of the travel of the illuminated light from the light source(s) in the optical system through the transmission window of the sensor and through the empty and filled reservoirs of the sensor, followed by collection of the reflected light by a detector.

FIG. 7A depicts a view of an embodiment of an optical bilirubin sensor with the lid removed.

FIG. 7B depicts a view of an embodiment of an optical bilirubin sensor with the lid attached.

FIG. 8A depicts an exploded, simplified not to scale longitudinal cross-sectioned view of an embodiment of an assembled sensor taken along the lines A-A′ of FIG. 7A.

FIG. 8B depicts the sensor of FIG. 7A in an assembled state.

FIG. 9A is a cross-sectional view of the sensor of FIG. 8B taken along the line A-A′ having a fluid sample applied thereto and illuminated by light from an embodiment of an optical system.

FIG. 9B is a cross-sectional view of the sensor of FIG. 8B taken along the line A-A′ having a fluid sample applied thereto and illuminated by light from an embodiment of an optical system.

FIG. 10 depicts a flowchart of an embodiment of a process for measuring bilirubin in a sample.

FIG. 11 depicts a bilirubin calibration curve used to calculate bilirubin concentration from sensor reflectance values.

FIG. 12 depicts a graph showing experimental bilirubin measurements compared to reference bilirubin measurements.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Disclosed in the present application are a sensor and methods for analyzing a liquid sample for bilirubin. In some embodiments, the sample is a vertebrate or mammalian blood sample, and the sample is placed on the sensor of the present disclosure, the sensor being readable using an analyzer. In some embodiments, the sample is analyzed for bilirubin concentration and results may be provided to a user in milligrams of bilirubin per deciliter of sample (mg/dL). In some embodiments, the sample is analyzed for bilirubin concentration using an optical measurement. Common components in whole blood such as hemoglobin or erythrocytes can inhibit the accurate optical measurement of serum bilirubin levels. In particular, hemoglobin demonstrates high absorbance at the same optical wavelengths as bilirubin. In some embodiments, the apparatuses and methods of the present disclosure do not require pretreating a whole blood liquid sample to remove or alter hemoglobin or erythrocytes prior to introduction of the sample to a sensor.

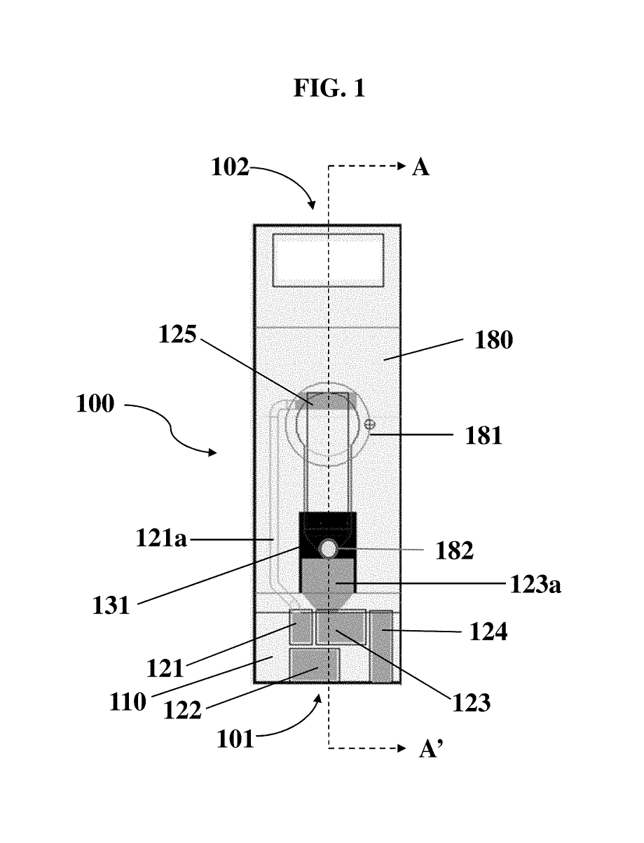

FIG. 1 depicts an embodiment of an optical bilirubin sensor configured to receive a liquid sample and facilitate analysis bilirubin levels in the sample. The sensor 100 is generally rectangular in shape and may comprise a base layer 110 and a lid layer 180 disposed on the base layer. Lid layer 180 includes a sample inlet 181 and a vent 182, each formed as holes that extend through a thickness of lid layer 180. In some embodiments, other layers may be disposed between base layer 110 and lid layer 180. Sensor 100 may further comprise an overall length dimension measured between the first end 101 and second end 102 along a line perpendicular to first end 101; an overall width dimension, measured along first end 101 or second end 102; and an overall thickness dimension, measured between a top surface of lid layer 180 and a bottom surface of base layer along a line normal to a top surface of lid layer 180. In some embodiments, the overall length dimension is 1.72″, the overall width dimension is 0.55″, and the overall thickness dimension is 0.031″. It will be understood by one of skill in the art, according to the principles and embodiments presently disclosed, that other dimensions are possible and within the scope of the present disclosure. For example, in some embodiments the overall length dimension is between about 0.5″ and about 6″, the overall width dimension is between about 0.25″ and about 3″, and the overall thickness is between about 0.005″ and about 0.5″; however, other sizes outside of these ranges are possible and contemplated. Further, it should be noted that other shapes, besides rectangular, may be used according to the principles and subject matter presently disclosed. In some embodiments, the dimensions of the sensor may correspond to a sensor support structure 220 and a sensor port on an analyzer 200 which will be described in greater detail below.

As depicted in FIG. 1, a first end 101 includes a plurality of contacts 121-124. In some embodiments, contacts may be as follows: a working electrode contact 121, sensor ID electrode contract 122, counter electrode contact 123, and a sensor insertion contact 124. In some embodiments, the contacts 121-124 are disposed on first end 101 of sensor 100 on an upper surface of base layer 110, and are exposed such that upon insertion of sensor 100 into a sample port of an analyzer, the contacts 121-124 make physical contact with corresponding contacts in the analyzer forming an electrical connection between sensor 100 and the analyzer. In some embodiments, the contacts 121-124 and trace 121a are silver. The silver layer can be printed on to base layer 110 using a silver-containing material screen. In some embodiments, the contacts and electrical traces may be printed, etched, or otherwise deposited on the base layer 110. Working electrode contact 121 is in electrical communication with trace 121a, the trace 121a extending generally away from the first end 101 of sensor 100 and toward the second end 102 of sensor 100. The working electrode contact 121 is in electrical contact with the working electrode 125 via trace 121a. Counter electrode 131, which may be carbon, is in electrical contact with counter electrode contact 123 via trace 123a. Although one configuration is depicted in FIG. 1 for the electric traces and the contacts, one of skill in the art will understand that a different contact order or trace configuration or trace and contact compositions can be used without departing from the scope of the present application.

As shown, some of the contacts disposed on first end 101 may be spaced back from the edge of first end 101, for example, working electrode contacts 121, and counter electrode contact 123. Other contacts may be disposed directly on the edge, for example, sensor ID electrode contact 122 and sensor insertion contact 124. Moreover, in some embodiments, the lengths and widths of the contracts may vary from contact to contact. In some embodiments, greater than five or fewer than four contacts may be used.

In operation, the working electrode contact 121 provides an electrical connection between the working electrode 125 and the analyzer which allow the analyzer to apply a voltage to the working electrode contact 121 and thus to the working electrode 125. the sensor ID electrode contact 122, when inserted into the analyzer, makes electrical contact with a corresponding contact in electrical contact structure 230 within the analyzer sensor port 210. The resulting electrical connection and electrical properties allow the analyzer 200 to recognize the optical sensor 100 as a bilirubin sensor. In some embodiments, the sensor ID electrode contact 122 provides additional information such as a manufacturing batch number or specific calibration information for the sensor 100 when it is connected to the sensor contacts in electrical contact structure 230 of the analyzer 200. In some embodiments, the counter electrode contact 123 provides a signal to the analyzer when the working electrode 125 and counter electrode 131 detects that the reflective layer is sufficiently wetted by the plasma. A reference voltage is generally applied to the counter electrode 131 from the analyzer. When a sample is applied to the sensor 100, the sample contacts the counter electrode 131, the conductivity of the sample alters a voltage or current or resistance measured in the analyzer based on the signal from the counter electrode 131.

In some embodiments, the sample contacts the counter electrode 131 and the working electrode 125. The conductivity of the sample can allow a current to flow through the sample between the counter electrode 131 and the working electrode 125, based on a reference voltage applied to the counter electrode. The change in current or resistance sensed between the counter electrode 131 and the working electrode 125 measured in the analyzer can be used to generate a signal that the sensor has been wetted by the sample, and is ready for analysis.

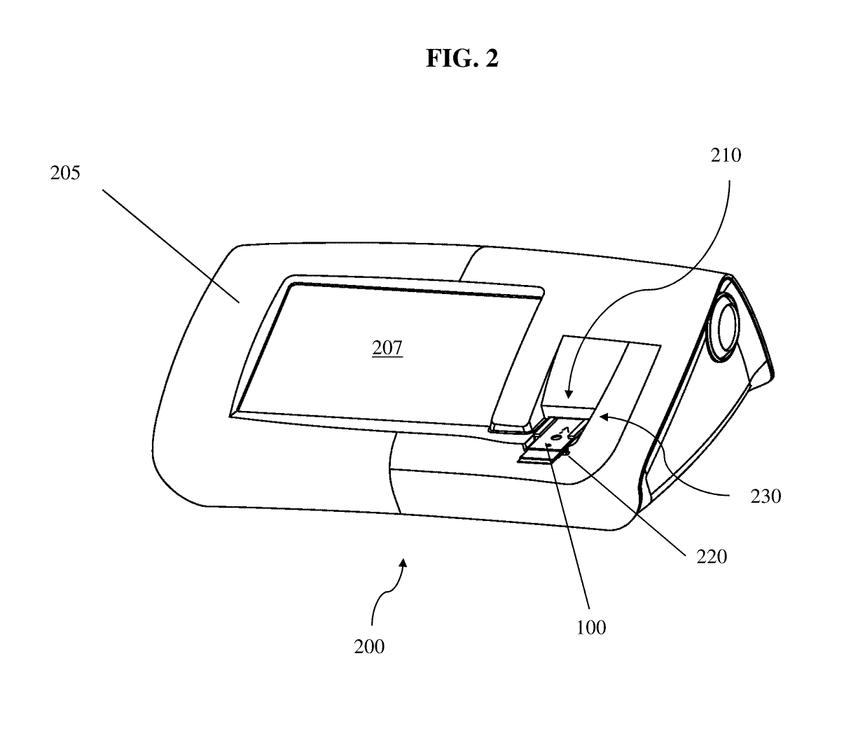

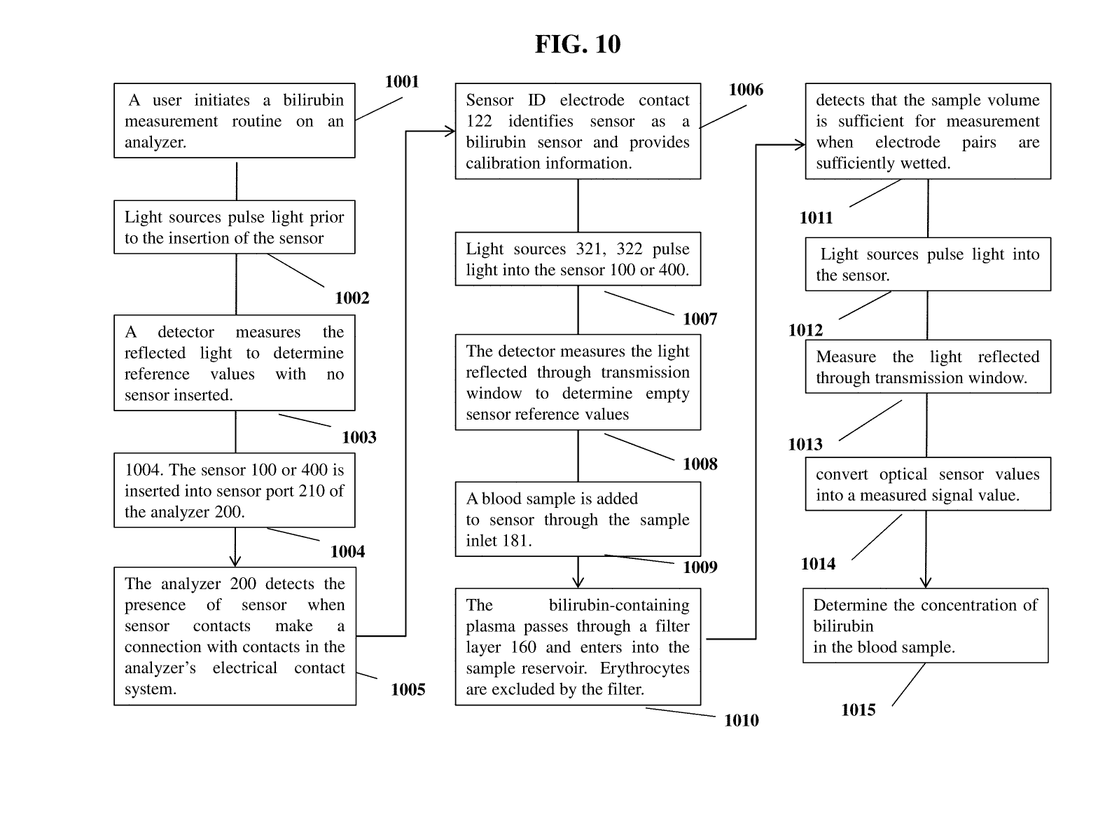

FIG. 2 depicts an embodiment of an analyzer 200 configured to receive and analyze a sample in an optical bilirubin sensor 100. Sensor 100 is configured in size and shape to be placed onto a sensor support structure 220 and inserted into a sample port 210 on an analyzer 200, wherein the sample port 210 has a compatible geometry configured to receive first end 101 of sensor 100. In some embodiments, the cross section of the sensor 100, the sensor support structure 220, and the sample port 210 are substantially rectangular as will be described in greater detail below. In some embodiments, sensor 100 is configured so that second end 102 remains exposed when first end 101 has been inserted into the sample port 210 of the analyzer. This configuration can allow a user to introduce the liquid sample to the sample port 181 of sensor 100 after sensor 100 has been inserted into the analyzer. The analyzer 200 may include a housing 205 configured in size and shape to be used on a tabletop or lab bench. In some embodiments, the housing 205 may be configured for hand held use. Housing 205 includes a display 207 that displays instructions and sample results to an operator. In some embodiments, the display 207 is an interactive display, such as a touch screen, which enables an operator to view, set, or select various analysis parameters and view sample results. In some embodiments, the analyzer 200 comprises an input device, such as a keyboard, soft or hard buttons, a mouse, or any other suitable input device which allows an operator to interact with the analyzer 200. An exemplary bilirubin measurement routine on an analyzer 200 is discussed further below in conjunction with FIG. 10.

A user initiates a bilirubin measurement routine on an analyzer 200. Light source(s) 321, 322 pulse prior to the insertion of a sensor. The sensor is placed on sensor support structure 220 for insertion into sensor port 210. The analyzer 200 detects the presence of sensor 100 when sensor insertion contact 124 makes an electrical connection with sensor contacts in electrical contact structure 230. As discussed above, the sensor ID electrode contact 122 identifies the sensor and provides calibration information.

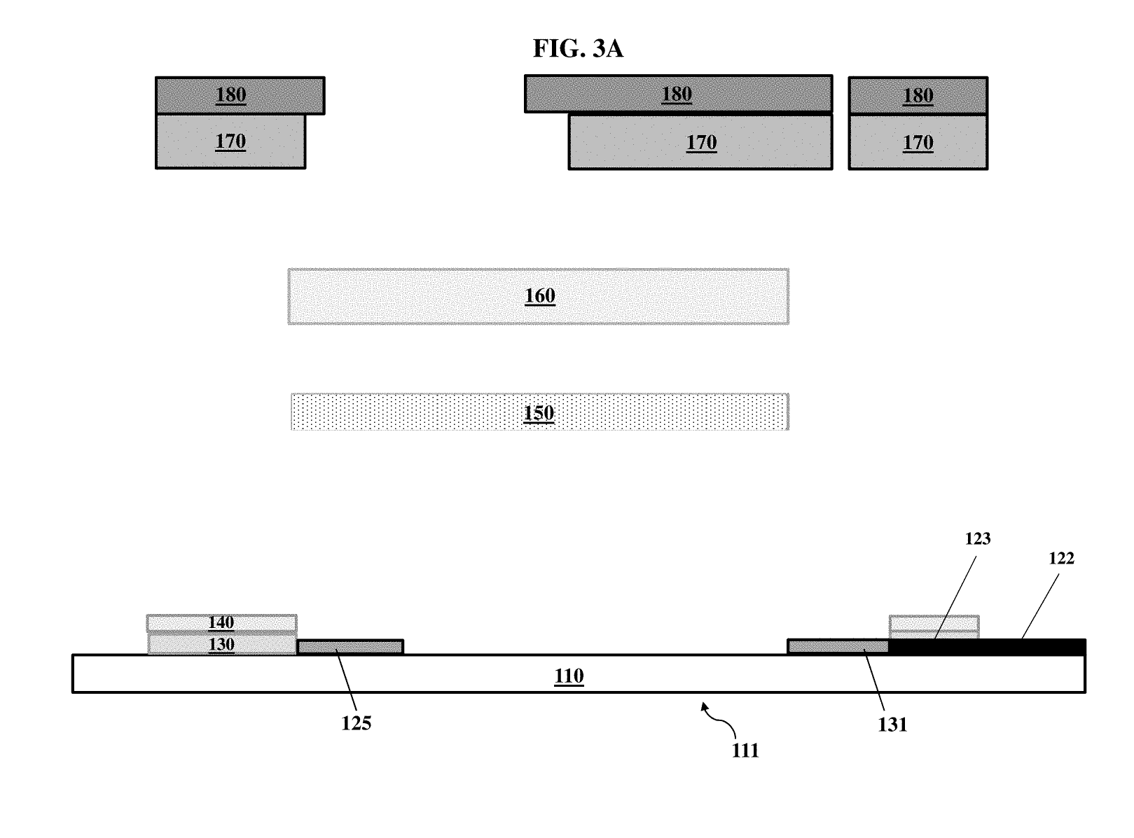

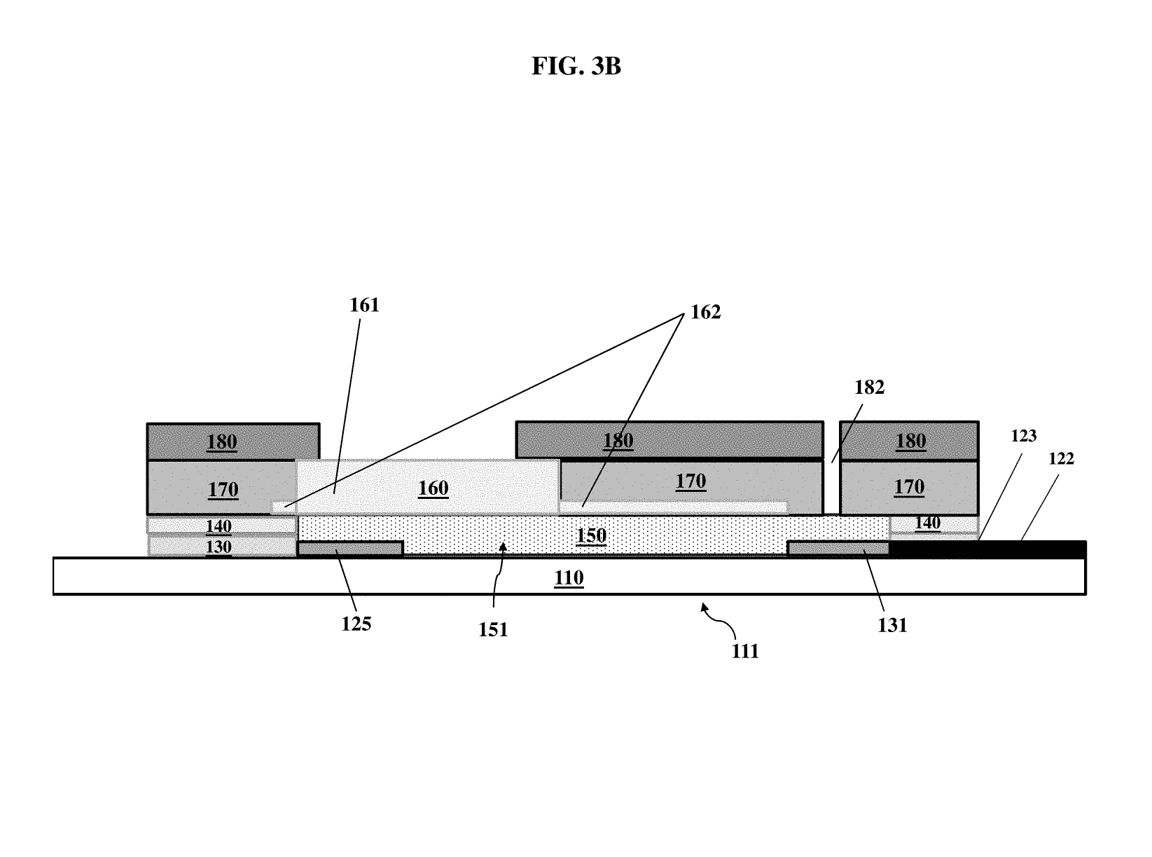

FIGS. 3A and 3B depict simplified (not to scale) exploded longitudinal cross-sectioned views of embodiments of an assembled sensor 100 taken along the lines A-A′ shown in FIG. 1. The illustrated layers in 3A can be stacked to form sensor 100, as shown in 3B. Sensor 100 may comprise a base layer 110, a dielectric layer 130, a reflective layer 150, a filter layer 160, a spacer layer 170, and a lid layer 180. A person of skill in the art will be aware that additional layers may be added as needed, such as carbon or silver layers. A thin layer of adhesive 140 may be applied between each successively stacked layer, bonding the layers together to form sensor 100. In some embodiments, each layer of adhesive is approximately 0.001 inches thick, although it will be understood by one of skill in the art that different thicknesses may be used. In some embodiments, bonding methods other than adhesive may be used, or sensor 100 may be manufactured or formed as a unitary piece, either through printing, molding, or other suitable manufacturing process.

Each layer of sensor 100 will now be described in greater detail with reference to FIG. 3A, which depicts embodiments of each of the layers separately for convenience and ease of description. In some embodiments, base layer 110 is generally rectangular in shape having a length of approximately 1.72″ a width of approximately 0.55″, and a thickness of 0.01″; it will be understood by one of skill in the art, however, that other dimensions for the base layer may be used. In some embodiments the length of base layer 110 extends beyond the other layers in a longitudinal direction, or along the length of the base layer 110, with the extended ends of base layer 110 forming the first end 101 and second end 102 of sensor 100.

Base layer 110 may comprise a transparent substrate that permits optical signals to pass there through. In some embodiments, the base layer 110 is formed entirely of a transparent material. In some embodiments, the base layer 110 is only partially comprised of a transparent material, the transparent material forming a transmission window 111 through the base layer 110 to allow for optical interrogation of a sample. In some embodiments, the transmission window is disposed between two separate electrodes, the working electrode 125 and the counter electrode 131 along a longitudinal axis of base layer 110 (see FIG. 1). The optically transparent material of base layer 110 or of the transmission window 111 may be formed from plastic, glass, sapphire, or other suitable material that permits at least light of wavelengths discussed below to be transmitted there through. In some embodiments, at least the transmission window 111 of the base layer 110 is made from polycarbonate or polyester. In some embodiments of base layer 110 a hard-coated, optical grade polycarbonate with a gloss finish is used for the transmission window 111.

The dielectric layer 130 is also disposed on top of base layer 110. The dielectric layer 130 may be an electrically insulating material, for example, a polymeric material. The dielectric layer 130 may serve to protect and isolate the active surfaces of the contacts 121-124 from the sample 190 as it flows into the reservoir of the reflective layer 150. In some embodiments, the dielectric layer 130 may also form the channel for the deposition of the reflective layer 150 as it provides for the outer bounds of the reflective layer 150. Modulating the height of the dielectric layer may also allow for increased or decreased thickness of the reflective layer as is necessary. The spacing of the dielectric layer may also allow for enhanced or decreased volume of the sample reservoir in the reflective layer 150 as is necessary.

The reflective layer 150 is disposed on an upper surface of base layer 110. In some embodiments, the reflective layer is disposed on all or a portion of the base layer 110. In some embodiments, reflective layer 150 is disposed on the transmission window 111 of the base layer 110 to allow reflection of light passing through the transmission window 111 during analysis. Reflective layer 150 may be generally rectangular in shape with a width less than or equal to the width of the base layer 110 and a length less than the length of base layer 110. One of skill in the art will understand according to the present disclosure that the reflective layer 150 may be round, square, diamond, or any shape suitable for use with the transmission window 111. One of skill in the art will also understand according to the present disclosure that various thicknesses may be used, for example, thicknesses of 0.0001″, 0.0005″, 0.001″, 0.005″, 0.010″, or any thickness there between. As will be described in greater detail below with regard to FIG. 3, the thickness of the reflective layer 150 affects the path length of light traveling through the sample and affects the amount of light available for detection. The reflective layer 150 is configured in size and shape to surround the electrodes 125 and 131 when the electrodes are disposed on base layer 110. A person of skill in the art, guided by the present disclosure, will understand how to vary the dimensions of the reflective layer 150 to accommodate different sample volumes and different path lengths of light.

In some embodiments, the reflective layer 150 is defined at its lateral edges by the adhesive and dielectric layers at its horizontal edges, by the base layer 110 at its bottom surface, and by the filter 160 and spacer layers 170 at its top surface. The reflective layer 150 also comprises at least a portion of the reservoir for the sample following its passage through filter layer 160. The thickness of the dielectric layer 130 along with the thickness of the adhesive layer 140 that binds the dielectric layer 130 to the adjacent layers may define the depth of the reflective layer, the depth of which impacts the sensor's ability to be used for optical bilirubin measurement given that insufficient thickness of the reflective layer may prevent sufficient passage of light through the sample for accurate optical bilirubin measurement. In some embodiments, the deposition method used for reflective layer 150 may determine the thickness of the reflective layer. In some embodiments, at least a portion of the reflective layer comprises a porous, reflective deposited material. The porous reflective deposited material can be a bilirubin binding material. In some embodiments, the porous reflective deposited material comprising the reflective layer 150 is a sprayed, sputtered, printed, or otherwise deposited ink formulation with a reflector. In some embodiments, the porous reflective deposited material 1 may be made with a material that can absorb the liquid sample and whose reflectance changes as the sample is absorbed. In some embodiments the porous reflective deposited material of the reflective layer 150 is a barium sulfate reflective material. The barium sulfate reflective material can comprise a gelatin, a cationic copolymer, and/or other components as necessary to act as a bilirubin binding material and to provide reflectance signals to the analyzer 200. The barium sulfate reflective material can be similar to those described in U.S. Pat. Nos. 4,069,017; 4,412,005; and U.S. Pat. No. 4,788,153 In some embodiments, a material that binds bilirubin may be sprayed, sputtered, printed, or otherwise deposited on the reflective layer 150. In some embodiments, the bilirubin binding material is a cationic copolymer and gelatin. The depth of the reflective layer 150, should be sufficiently thick so as to ensure that most of the light is reflected back out of sensor 100. If the porous reflective layer 151 lacks sufficient depth, insufficient light will be reflected out of sensor 100 and optical measurements will be inaccurate. Further, as some embodiments may require a minimum amount of light absorbance or absorption, insufficient depth or thickness of the porous, reflective deposited material may also produce inaccurate optical measurements. This effect can be minimized by ensuring that porous reflective material layer 151 contains sufficient depth or thickness, for example, 0.0005″ or 0.001″.

The filter layer 160 is disposed between the reflective layer 150 and the spacer layer 170. The filter layer 160 is positioned between the reflective layer 150 and the spacer layer 170 so as to align, at least in part, with a sample inlet 181, formed in the lid layer 180, which will be described in greater detail below. The filter layer 160 comprises a filter that inhibits the passage of molecules and/or compounds that hinder an accurate optical measurement of bilirubin in a liquid sample. Some constituents in whole blood, such as hemoglobin have significant absorbance of optical signals. The light absorbance of the whole blood constituents can mask or interfere with a measurement of another analyte of interest, such as bilirubin. In some embodiments, the filter may inhibit the passage of cells (e.g. erythrocytes) which may contain quantities of molecules, such as hemoglobin, which can interfere with optical measurements of bilirubin. In some embodiments, the filter layer 160 has a large surface area to ensure the efficient passage of plasma into the portion of the reservoir in the reflective layer 150. To ensure that sufficient plasma volume has been reached, electrodes 125 and 131 are disposed on base layer 110 and oriented so as to detect when the reflective layer is wetted by plasma. This may be particularly useful for liquid samples with an appreciable concentration of contaminating molecules that may clog the filter, such as neonatal blood samples with high erythrocyte concentrations. In some embodiments, a keyhole filter configuration may prevent the leakage of contaminants such as erythrocytes into the reflective layer 150. In some embodiments, firm pressure is provided by the spacer layer 170 to prevent the leakage of contaminating molecules and to aid in the wicking of the plasma toward the transmission window. In some embodiments, areas of the filter layer 160 are compressed by pressure placed upon the lid 180 and/or spacer layer 170 to prevent leakage of erythrocytes into the portion of the reservoir in the reflective layer or the portion of the reservoir in the filter layer and to enhance wicking of the blood plasma into the transmission window as demonstrated in FIG. 3B. This compression also prevents the passage of erythrocytes into the portion of filter layer 160 above the transmission window, minimizing the risk of potentially inaccurate readings due to the interaction of erythrocytes with light emitted into the sensor 100. Thus, in some embodiments, the filter layer 160 is separated into compressed filter region 162 and uncompressed filter region 161. The filter layer may also comprise at least a portion of the sample reservoir. In some embodiments, at least a portion of the light input through the transmission window 111 may pass through the portion of the sample reservoir in the reflective layer 150 and enter into the portion of the sample reservoir in filter layer 160. In some embodiments, a portion of the light entering into the portion of the reservoir in the filter layer is reflected out transmission window 111 and collected.

The spacer layer 170 is disposed on top of the filter layer 160. The spacer layer 170 may be made from white polyester or any other suitable material. In some embodiments, a suitable material may be one that can be used as a diffuse reflector. In some embodiments, the material may be hydrophilic, or coated with a hydrophilic substance. In some embodiments the spacer layer 170 is approximately 0.001, 0.005, 0.01, 0.15, 0.2 inches thick or more, or any thickness there between. The size, shape, or thickness of the spacer layer 170 and/or the adhesive layer 140 affects the volume of contiguous filter which is accommodated on the sensor, as during construction of the sensor 100, a portion of the spacer layer 170 is disposed directly on the filter layer 160, and will compress the portion of the filter layer 160, as shown in FIG. 2B. The size, shape, or thickness of the spacer layer 170 and/or the adhesive layer 170 may be used to determine the size, shape, or thickness of at least a portion of the filter layer 160. The size, shape, or thickness of the spacer layer 170 and/or the adhesive layer 140 may be modified to accommodate differing filters, to prevent erythrocyte leakage into the reflective layer 150, to prevent erythrocyte movement along the filter layer 160 to a position above the transmission window 111, or for any other suitable purpose. A person of skill in the art, guided by the present disclosure, will understand how to vary the size, shape, or thickness of the spacer layer 170 for these purposes.

A lid layer 180 is disposed on top of spacer layer 170. The lid layer 180 is configured in size and shape to have similar width and length dimensions as pacer layer 170. In some embodiments, the lid layer 180 is 0.001, 0.005, 0.01, 0.02 inches thick or more, or any value there between. Lid layer 180 may be comprised of a plastic or other suitable material. In some embodiments, lid layer 180 is coated with a hydrophilic substance so that the reservoir can be more easily filled with the sample 190. In some embodiments, lid layer 180 and/or spacer layer 170 may also be formed of a clear, transparent, or translucent material. The use of a clear, transparent, or translucent material may facilitate a visual indication to the user when the reservoir is filled. In some embodiments, lid layer 180 may be opaque so as to shield the optical measurements that will be discussed below from interference from ambient light. It will be noted, however, that a clear lid layer 180 and/or spacer layer 170 may be used and obtain an accurate optical measurement according to the present disclosure. The lid layer 180 provides an upper boundary on a sample reservoir within sensor 100 to prevent evaporation of the sample 190. Lid layer 180 also includes a sample inlet 181 and a vent 182 formed as voids extending through a thickness of the lid layer 180 as well as through the thickness of spacer layer 170 below. The relative positioning of the inlet 181 and vent 182 depicted in FIG. 3A and 3B is merely illustrative and one of skill in the art will appreciate that the positioning of the inlet 181 and vent 182 may vary without departing from the scope of the present disclosure. FIG. 3B depicts the same sensor 100 in an assembled state.

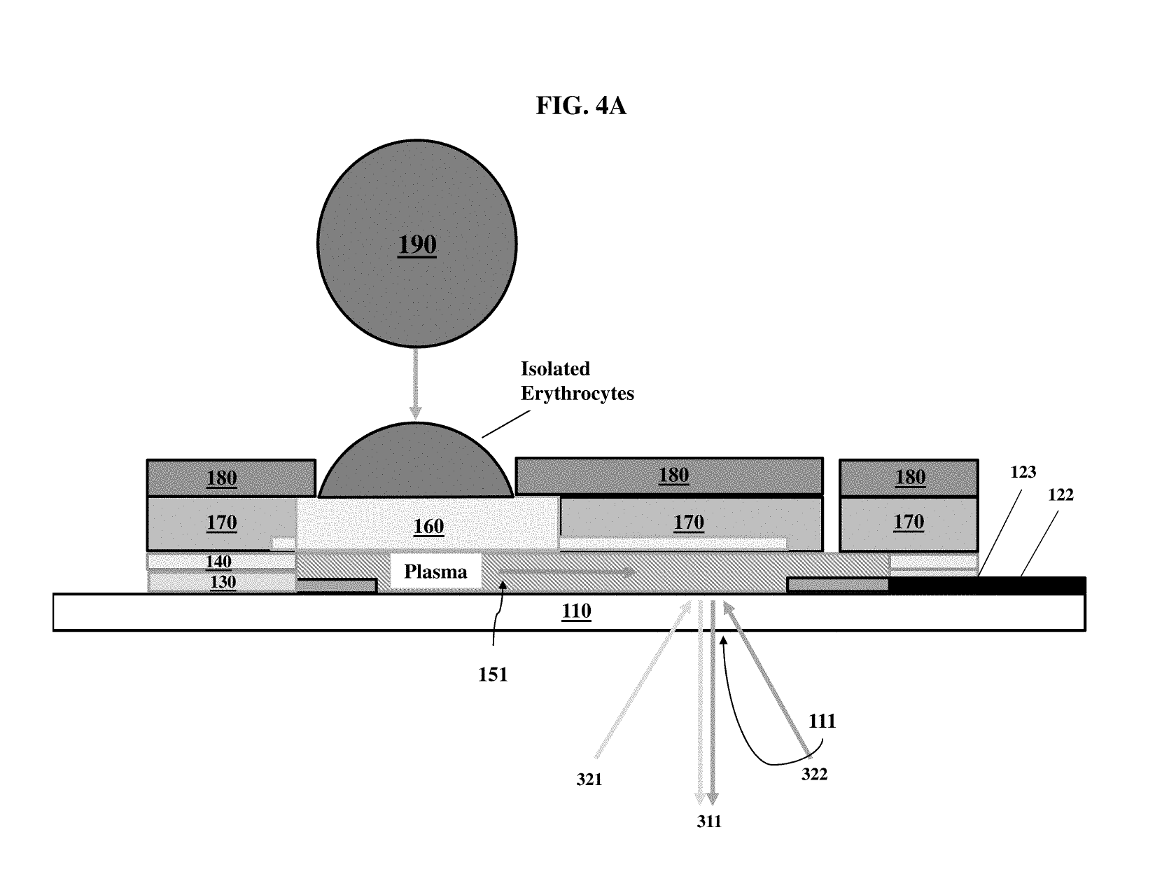

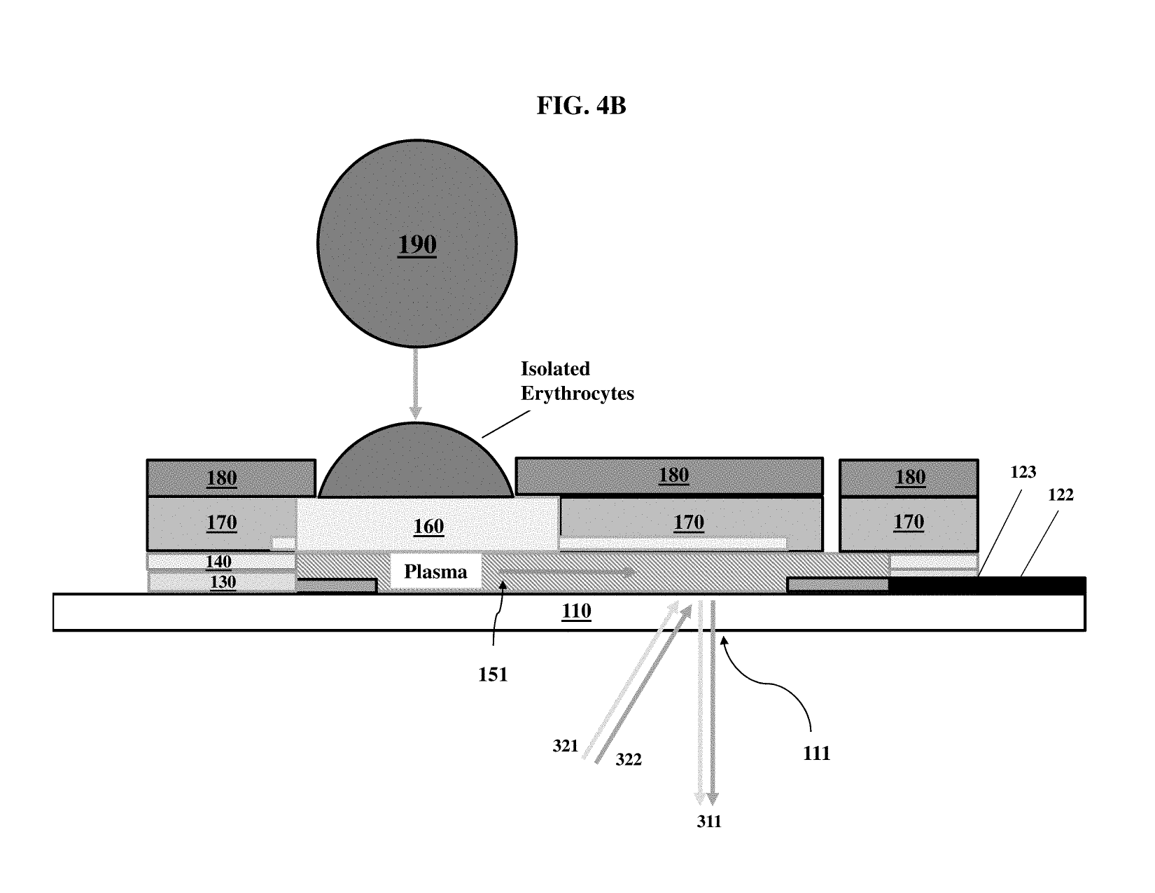

FIG. 4A depicts a simplified view of the operation of an embodiment of an optical bilirubin sensor taken along the line A-A′ with all layers combined. The sensor 100 is first inserted into a sensor port of the analyzer. The interaction between the analyzer and the sensor insertion contact 124 notifies the analyzer that a sensor has been inserted. Interaction between the analyzer and sensor ID electrode contact 122 identifies the sensor as a bilirubin sensor. Next, light sources 321 and/or 322 initiate alternating emissions of light at a target wavelength and a secondary wavelength to determine empty sensor reflectance to take a reference sample. In an embodiment disclosed in FIG. 4A, at least one light source is used to emit light at the target wavelength and at least one other light source is used to emit light at the secondary wavelength. In an embodiment disclosed in FIG. 4B, at least one light source is capable of emitting light at both the target and secondary wavelengths. The target wavelength is used to determine the analyte concentration, and the secondary wavelength to correct for the possible presence of a contaminant that may absorb at a similar wavelength, such as hemoglobin. In some embodiments discussed further below, light sources 321 and/or 322 may emit additional wavelengths or broad spectrum wavelength light, such as white light. Light from the light sources is directed through the base layer 110, such as through the transmission window 111. The light passes through the transmission window 111, impinges the reflective layer 150 and/or the filter layer 160, is reflected back through the transmission window, and is detected by the analyzer detector 310. In some embodiments, a bilirubin-free sample may be used for a reference sample. In some embodiments, a reference sensor may be used to establish reference or baseline measurements.

Following the measurement of the reference sample, the sample 190 to be analyzed is then introduced to the sensor 100 at sample inlet 181. The sample 190 may be a sample of whole blood. Sample 190 moves through the filter layer 160. The filter layer 160 excludes erythrocytes and other unwanted sample components. One of ordinary skill guided by this disclosure would recognize that any suitable type of filter or filtering material may be used to filter out undesirable sample components. The bilirubin containing plasma from sample 190 that has passed through filter layer 160 arrives in the reflective layer 150. The relatively large available filter surface area 160 allows for the necessary plasma volume to enter the reservoir. Neonates have a very high concentration of red blood cells that can clog smaller filter surface areas. The filter further comprises a compressed filter region 162 under the lid 180 and/or spacer layer 170 to seal the erythrocytes from moving around the edge, and aids in the wicking of the plasma into the transmission window. The void volume of the crushed filter or the sample reservoir is defined by the other sensor components, which may include the lid 180 and/or spacer layer 170. Capillary action facilitates the efficient wicking of plasma through the reflective layer 150 to the contacts.

Vent 182 is provided to prevent overfilling and to allow air to escape as the sample reservoir in the reflective layer 150 is filled. The thickness of dielectric layer 130 and the adhesive layer 140 may define the depth between the base layer 110 and the reflective layer 150. In some embodiments, the reservoir depth in the reflective layer 150 is approximately 0.004 inches deep. The filter, which may be setup in a keyhole configuration, prevents erythrocytes in the liquid sample 190 from passing through to the reservoir. Plasma containing an analyte of interest, such as bilirubin, passes through the filter and wicks into the reflective layer. The plasma wicking into the filter layer 160 and the reflective layer 150 contacts the working electrode 125 and the counter electrode 131. When the working electrode 125 and the counter electrode 131 is contacted with the plasma the analyzer identifies that the working electrode 125 and counter electrode 131 is wetted with the plasma, and initiates the measurement process. After a brief delay to ensure stabilization of the optical reflectance, the one or more light sources, 321 and/or 322, initiate alternating pulsing to determine the filled sensor reflectance at one, two or more distinct wavelengths. As discussed below, the light source(s) 321 and/or 322 may emit light at a single wavelength, two or more distinct wavelengths, or a broad spectrum of wavelengths of light. A person of skill in the art will recognize that any suitable wavelength, wavelengths, or spectrum of wavelengths may be used. A person of skill in the art will also recognize that additional light sources may be added or removed as necessary.

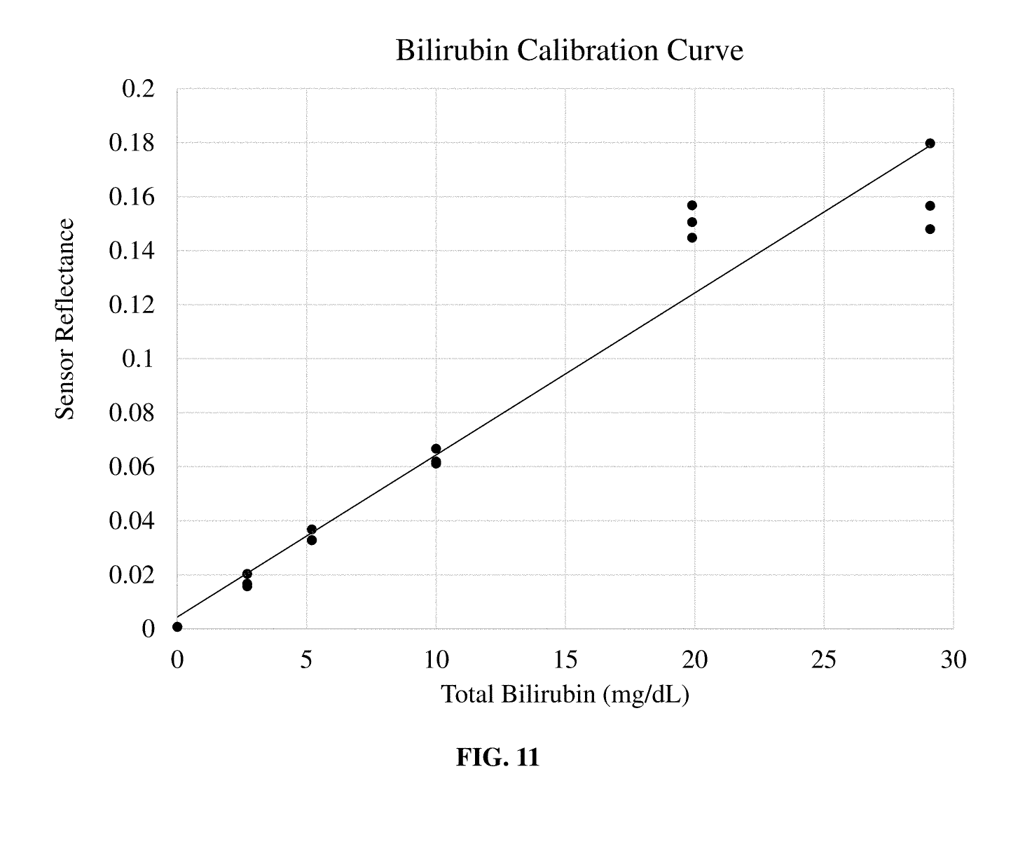

In some embodiments, the reflectance of the sample is then measured at both a target and secondary wavelength. After both wavelengths are measured, the reflectance of the secondary wavelength is subtracted from the target wavelength to eliminate the effect of contaminants. This corrected value is then compared to a bilirubin calibration curve and an analyte concentration is calculated. In some embodiments, the target wavelength is approximately 480 nm and the secondary wavelength is 525 nm. This calculation will be described in greater detail below in conjunction with Equation 1 and FIG. 11.

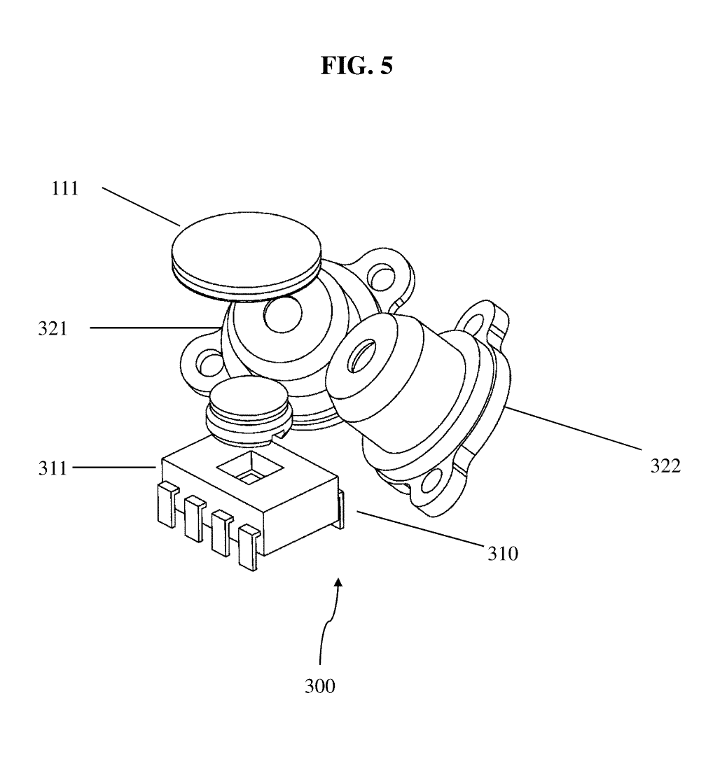

FIG. 5 depicts an embodiment of an optical system 300 that may be contained within the analyzer 200. The optical system comprises at least one light source capable of producing light at a target wavelength, a secondary wavelength, multiple distinct wavelengths, or a spectrum of wavelengths. In some embodiments, the optical system comprises at least two LED light source(s), 321 and 322, one of which produces light at a target wavelength and a second of which produces light at a secondary wavelength. A person of skill in the art will recognize that additional light source(s) may be used, such as at least one light source that may produce light at many distinct wavelengths, at least one light source that may emit a spectrum of wavelengths light, or any other suitable light source. Light from the light source(s) 321 and/or 322 pass through the transmission window 111 into the sample 190 and are reflected back out of the transmission window 111. The reflected light is collected by a collection lens 311 and enters into a detector 310. In some embodiments, the collection lens 311, the detector 310, or both may collect light at one or more distinct wavelengths. In some embodiments, the collection lens 311 and/or the detector may collect reflected light at all wavelengths. In some embodiments, the collection lens 311, the detector 310, or other components of the analyzer 200 may filter out light consisting of unnecessary or undesirable wavelengths. One of ordinary skill guided by this disclosure would recognize that any suitable method may be used to filter out light consisting of unnecessary or undesirable wavelengths. In some embodiments, the detector 310 may only determine the intensity of reflected light at one or more specific wavelengths. The detector 310 determines the intensity of the reflected light and sends a signal to the analyzer 200.

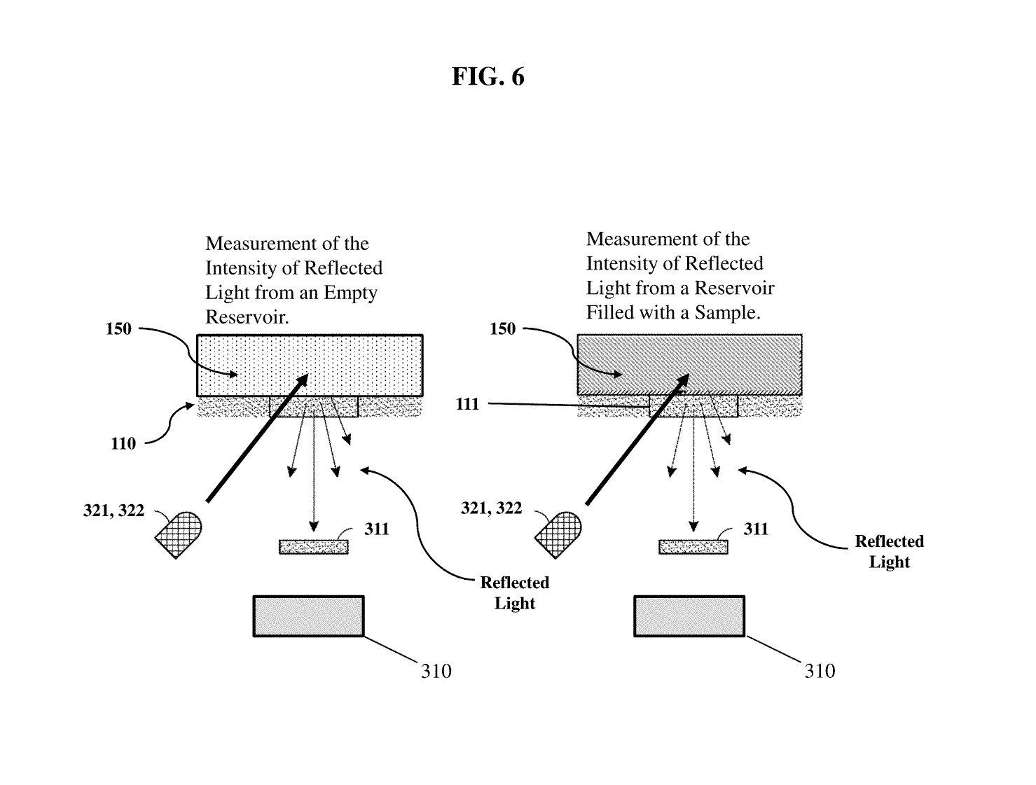

FIG. 6 is a depiction of the operation of the optical system 300 in the analyzer 200 used in FIG. 5 to measure the intensity of reflected light from an empty or filled reservoir. Light source(s) 321 and/or 322 pulse light at a target and secondary wavelength through the transmission window 111 in base layer 110 through a sample 190 in the reservoir of reflective layer 150. The light traveling from the light source 321 and/or 322 travels through the reservoir and is reflected outward through the transmission window and is collected by the collection lens 311 and detected by detector 310. The measured optical values may then be used by the analyzer 200 to determine the concentration of the analyte in the sample 190.

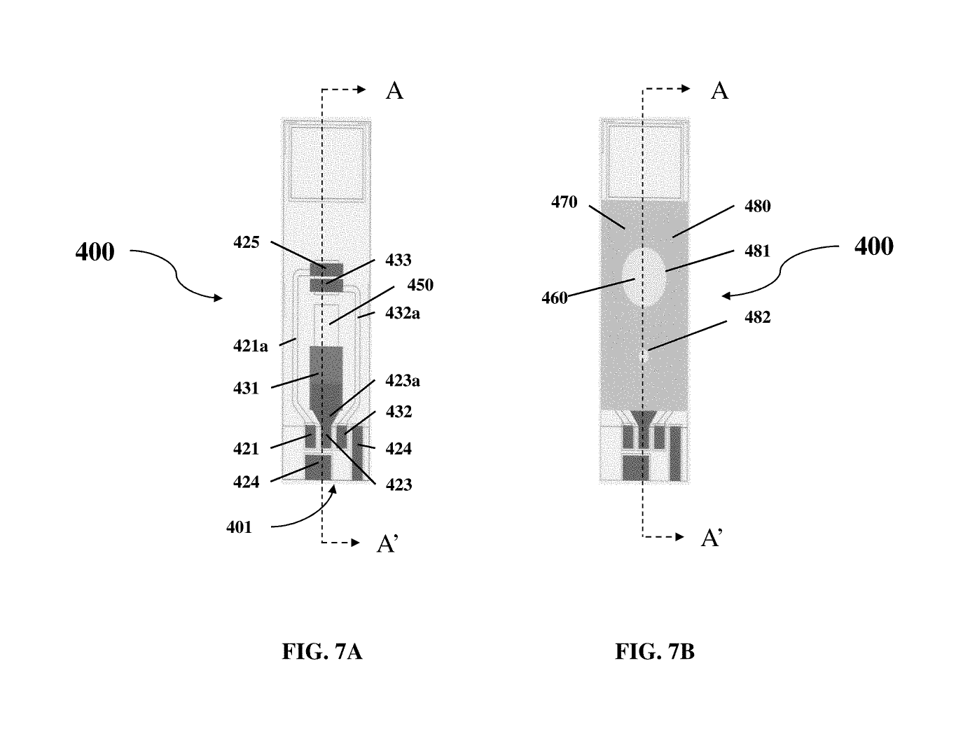

FIGS. 7A and 7B depict an embodiment of an optical bilirubin sensor 400. In some embodiments, the sensor 400 comprises a working electrode contact 421, sensor ID electrode contact 422, counter electrode contact 423, sensor insertion contact 424, and a reference contact 432 on a first end 401. In some embodiments, a reference trace 432a and a reference electrode 433 may be added. For ease of discussion, all other components in this second embodiment retain the functionality as equivalently numbered components as described in the sections provided above, working electrode contact 421 retaining the same functionality as working electrode contact 121 in the sensor 100 disclosed in FIG. 1. Other examples of components retaining the functionality of equivalently numbered components include traces 421a, 423a, and electrodes 425 and 431, which retain the functionality of traces 121a, 123a, and electrodes 125 and 131. By modifying the counter electrode contact 423 with its accompanying electrodes 425 and 431 and adding an additional reference contact 432 with an accompany trace 432a and reference electrode, additional plasma sensing capabilities have been added. In some embodiments, the reference electrode 433 allows for the detection of the initial plasma wetting as well as the detection of sufficient plasma volume for optical measurements to determine bilirubin concentration. The counter electrode contact 423 and electrodes 425 and 431 further provide a signal when the plasma volume has sufficiently filled the reflective layer such that optical measurements may be made. FIG. 7B depicts a top view of an embodiment of sensor 400 wherein a spacer layer 470, a filter layer 460, a lid 480, a sample application port 481 and a vent 482, are shown. The sample application port 481 is the portion of the sensor 400 to which the whole blood sample is applied. The vent 482 allows for air to escape as capillary action wicks the sample along the filter layer 460 and/or the reflective layer 450.

For example, when a sample is applied to the sample port 481, the sample flows through the filter layer 460, wherein erythrocytes are filtered out. The sample then flows into the reflective layer 450, wherein the sample comes into contact with working electrode 425 and reference electrode 433. When sufficient sample as contacted the working electrode 425 and the reference electrode 433, a circuit path is created between the working electrode 425 and the reference electrode 433. The referenced electrode is maintained at a voltage from the analyzer via the associated contacts in analyzer 200, and a current flows between the working electrode 425 and the reference electrode 433. The analyzer detects this current at the associated contacts on the sensor 400, and interprets the sensor 400 has having been wetted. As the sample continues to wick along the reflective layer, the sample will contact electrode 431. This will create a second current flow path between the reference electrode 433 and counter electrode 431. The analyzer 200 detects this current at the contact associated with the counter electrode contact 423, and determines that sufficient sample is above the transmission window 411, and that sample is ready to be measured.

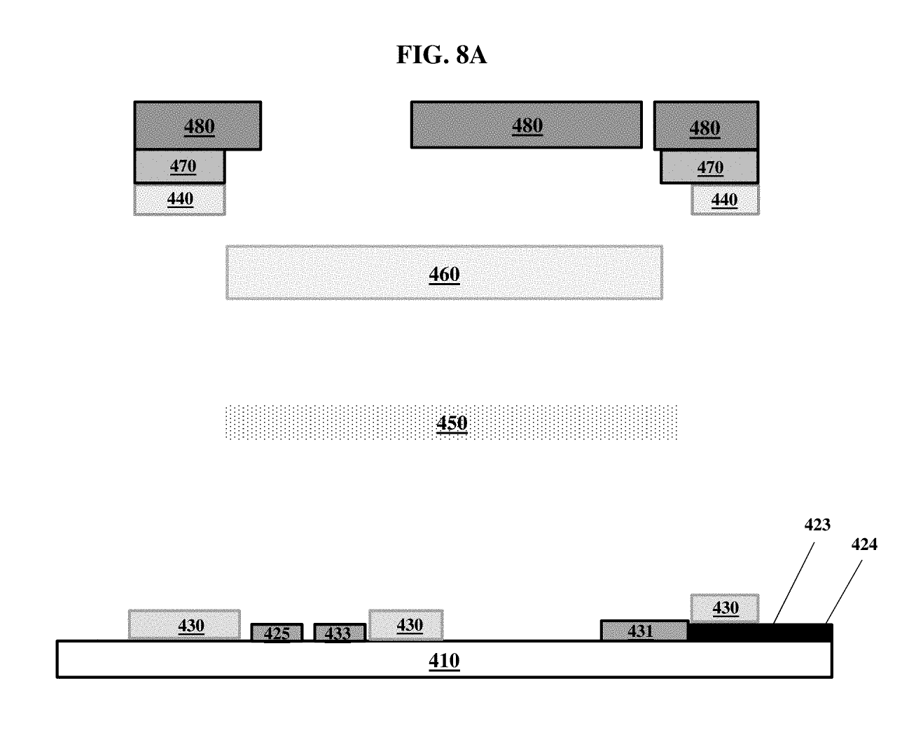

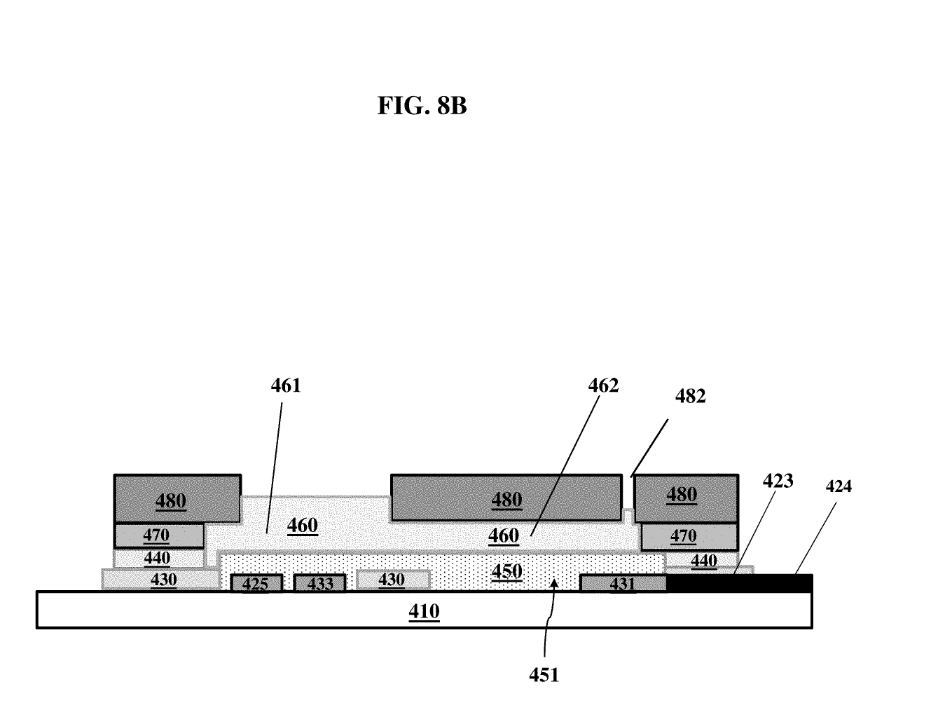

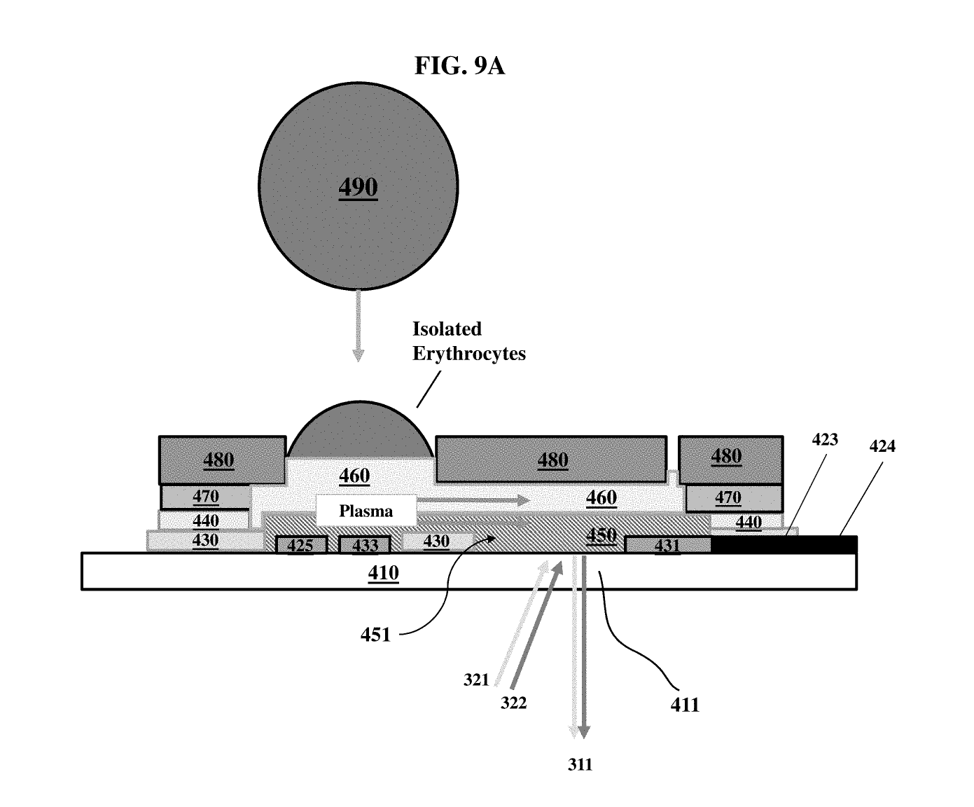

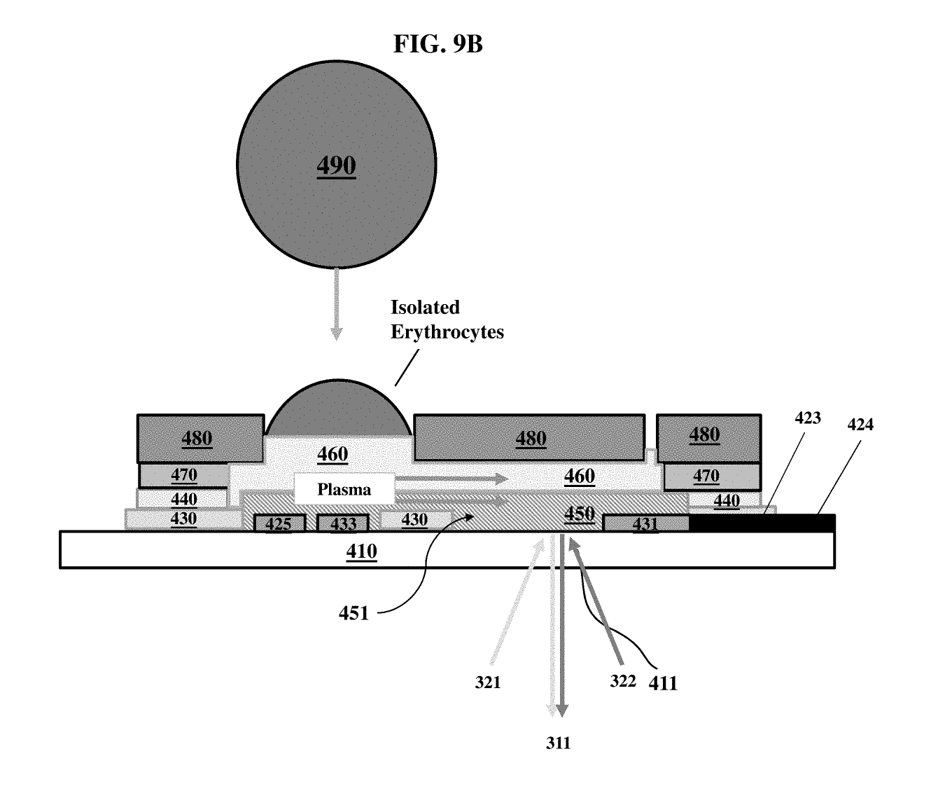

FIG. 8A depicts an exploded, simplified not to scale longitudinal cross-sectioned view of an embodiment of an assembled sensor taken along the lines A-A′ of FIG. 8. This figure demonstrates the addition of reference electrode 433 to sensor 400 in some embodiments. FIG. 8B depicts the sensor of FIG. 9A in an assembled state. This depiction demonstrates the compressed regions 462 of filter layer 460 compressed by lid 480 as well as areas of the filter layer 460 that are not compressed 461 by the lid 480. In some embodiments, the size, shape, or thickness of spacer layer 470 and/or adhesive layer 440 may interact with lid 480 to directly impact the pressure that lid 480 places on the filter layer 460 without direct interaction between spacer layer 470 and/or adhesive layer 440 with filter layer 160. The interaction between lid 480 and spacer layer 470 and/or adhesive layer 440 may also affect the size, shape, or thickness of compressed filter region 461. This may further facilitate compression consistency for compressed filter region 461 during the assembly or manufacturing process. In some embodiments, the size, shape, or thickness of spacer layer 470 and/or adhesive layer 140 affect compressed filter region 461 without

FIG. 9A is a cross-sectional view of the sensor of FIG. 8 taken along the line A-A′ having a fluid sample 490 applied thereto and illuminated by light from an embodiment of an optical system 300. In an embodiment demonstrated herein, light sources 321, 322 from different directions may be used to produce light at a targeted and secondary wavelength. As previously discussed, optical system 300 may include a single light source 321 or multiple light sources, 321, 322, or more as necessary. The one or more light sources may emit a single wavelength, multiple wavelengths, or a spectrum of wavelengths of light. The reflected light is collected by a detector 310 in the optical system 300. In some embodiments, the collection lens 311, the detector 310, or both may collect light at one or more specific wavelengths. In some embodiments, the collection lens 311, the detector 310, or other components of the analyzer 200 may filter out light from unnecessary or undesirable wavelengths. In some embodiments, the detector 310 may only determine the intensity of reflected light at one or more specific wavelengths.

FIG. 9B is a cross-sectional view of the sensor of FIG. 8 taken along the line A-A′ having a fluid sample 490 applied thereto and illuminated by light from an embodiment of an optical system 300. In an embodiment demonstrated herein, at least one light source from a certain direction is used to produce light at a targeted and secondary wavelength. The reflected light is collected by a detector 310 in the optical system 300. A person of skill in the art guided by the current disclosure will recognize that the number and type of light sources may be varied and that a single wavelength, multiple wavelengths, or broad spectrum wavelengths may be used. Any suitable light source, wavelength or wavelengths, or spectrums of wavelengths may be used. For example, in some embodiments, the light sources 321, 322 can be a broad spectrum emitter emitting a broad spectrum of light, such as white light, or any other desired portion of the electromagnetic spectrum. The detector 310 can be configured to measure the reflected wavelengths. The detector 310 may be configured to read only the desired wavelengths from the broad spectrum which are useful for determining bilirubin concentration. In some embodiments, the detector 310 may be connected to a processor which receives signals from the detector 310 corresponding to the received wavelengths, and the processor is configured to separate the wavelengths of interest for measuring bilirubin concentration.

FIG. 10 depicts a flowchart presenting an embodiment of a bilirubin measurement routine 1000. In block 1001, a user initiates a bilirubin measurement routine on an analyzer 200. This can be done by selecting an appropriate button on the analyzer or by instructing the analyzer to run a specific routine.

The process moves to block 1002, where the initiated analyzer 200 send a signal to light source(s) 321 and/or 322 to pulse light prior to the full insertion of a sensor 100 or 400.

The process moves to block 1003, wherein the analyzer 200 detects the reflected light from the light sources 321, 322 in the detector 310 and uses the reflected light to set a background reflectance, or a reference reflectance. For example, the analyzer 200 pulses the light sources 321, 322, and reflected light travels through the transmission window 111, through the collection lens 311, and into the detector 310 to provide reference “no sensor inserted” optical values (e.g. B_{Dark} and G_{Dark}). The definitions of exemplary optical value labels such as B_{Dark} and G_{Dark} are defined below under Equation 1. In some embodiments, optical values at additional, alternative, or spectrums of wavelengths may be measured. In some embodiments, the light source(s) may pulse light prior to the full insertion of the sensor 100 or 400 into the sensor port 210 of the analyzer 200 as the sensor 100 or 400 is placed on the sensor support structure 220.

The process moves to block 1004, wherein the sensor 100 or 400 is inserted into the sensor port 230. In block 1005, the analyzer 200 detects the presence of sensor 100 or 400 when sensor insertion contact 124 and other contacts make an electrical connection with sensor contacts in electrical contact structure 230. As the sensor 100 or 400 is inserted, the sensor insertion contact 124 can complete a circuit with the electrical contact structure 230. The analyzer 200 receives a current signal from the completed circuit, and this signal indicates to the analyzer 200 that a sensor has been inserted.

In block 1006, sensor ID electrode contact 122 identifies the sensor as a bilirubin sensor and may provide calibration information. In block 1007, the light source(s) 321 and/or 322 pulse light into the sensor 100 or 400 through the transmission window 111. In block 1008, the light is reflected through the transmission window 111, collected by the collection lens 311, and enters the detector 310 to determine optical values for an empty sensor (e.g. B_{Empty} and G_{Empty}). In some embodiments, the empty sensor may not contain a sample. In some embodiments, the empty sensor may comprise a bilirubin-free sample.

In block 1009, a blood sample 190 is added to sample inlet 181 on top of filter layer 160. The process moves to block 1010, wherein the bilirubin-containing plasma passes through a filter layer 160, where the erythrocytes are filtered out or retained in the filter layer 160, and the filtered sample, or plasma, moves into the reflective layer 150. The plasma contacts the working electrode 425 and the reference electrode 433 and generates a signal, which the analyzer interprets as the sensor 400 is wetted.

The process moves to block 1011, wherein the filtered sample or plasma continues to wick along the reflective layer 450, where the sample contacts counter electrode 431. This creates a current flow path, as described elsewhere herein, which generates a current and signal which the analyzer 200 interprets as the sample being ready for measurement. For example, when sufficient sample is in the reflective layer 450 for optical measurements, working electrode contact 123 and the counter electrode 131 (or their counterpart electrodes 425 and 431) send a signal to the analyzer 200.

The process moves to block 1012 wherein the light sources 321, 322 emit light, which passes through the transparent window 411 and into the reflective layer 450, where the sample is located. The light reflects and/or scatters through the reflective layer 450. A portion of the light can be absorbed by the bilirubin within the reflective layer 450.

The process moves to block 1013, wherein the reflected light from the reflective layer 450, and possibly the filter layer 460 depending on the scattering of the light within the reflective layer 450, is reflected back through the transmission window 111, 411, and are detected in the detector 310. The detector 310 measures the intensity of the light reflected through transmission window 111 to determine optical values for the sample 190, (e.g., B_{Sample} and G_{Sample}).

The process moves to block 1014, wherein the analyzer 200 utilizes the relevant optical sensor values and inputs them into Equation 1 or an equivalent equation or formula to calculate a measured signal value. In block 1015, the measured signal value is input into an equation derived from a bilirubin calibration curve to determine the concentration of bilirubin in the blood sample 190, as will be described herein below.

FIG. 11 depicts a bilirubin calibration curve for converting corrected optical measurement values from an analyzer to calculate bilirubin concentrations. Equation 1 provides a method for converting optical data into a measured signal value that can be used with the bilirubin calibration curve to determine the bilirubin concentration for a liquid sample. In some embodiments, this equation includes an additional variable, X_{Instrument,} that may be used to calibrate the measured signal values based on the instrument used.

Equation for Converting Optical Data into a Measured Signal Value

### Measured Signal

$$\begin{matrix}Measured Signal = \frac{{[ 1 - \frac{( {B}_{Sample} - {B}_{Dark} )}{( {B}_{Empty} - {B}_{Dark} )} ]}^{2}}{[ 2 * \frac{( {B}_{Sample} - {B}_{Dark} )}{( {B}_{Empty} - {B}_{Dark} )} ]} - ( \frac{{[ 1 - \frac{( {G}_{Sample} - {G}_{Dark} )}{( {G}_{Empty} - {G}_{Dark} )} ]}^{2}}{[ 2 * \frac{( {G}_{Sample} - {G}_{Dark} )}{( {G}_{Empty} - {G}_{Dark} )} ]} ) * {X}_{Instrument} \\ {B}_{Dark} = Instrument Optical Value with no \\ sensor inserted at Blue wavelength \\ {B}_{Empty} = Instrument Optical Value with \\ sensor inserted at Blue wavelength \\ {B}_{Sample} = Instrument Optical Value with sensor \\ inserted , sample applied at Blue wavelength \\ {G}_{Dark} = Instrument Optical Value with no \\ sensor inserted at Green wavelength \\ {G}_{Empty} = Instrument Optical Value with \\ sensor inserted at Green wavelength \\ {G}_{Sample} = Instrument Optical Value with sensor \\ inserted , sample applied at Green wavelength \\ {X}_{Instrument} = Instrument dependent calibration factor . & \end{matrix} \qquad Equation 1$$

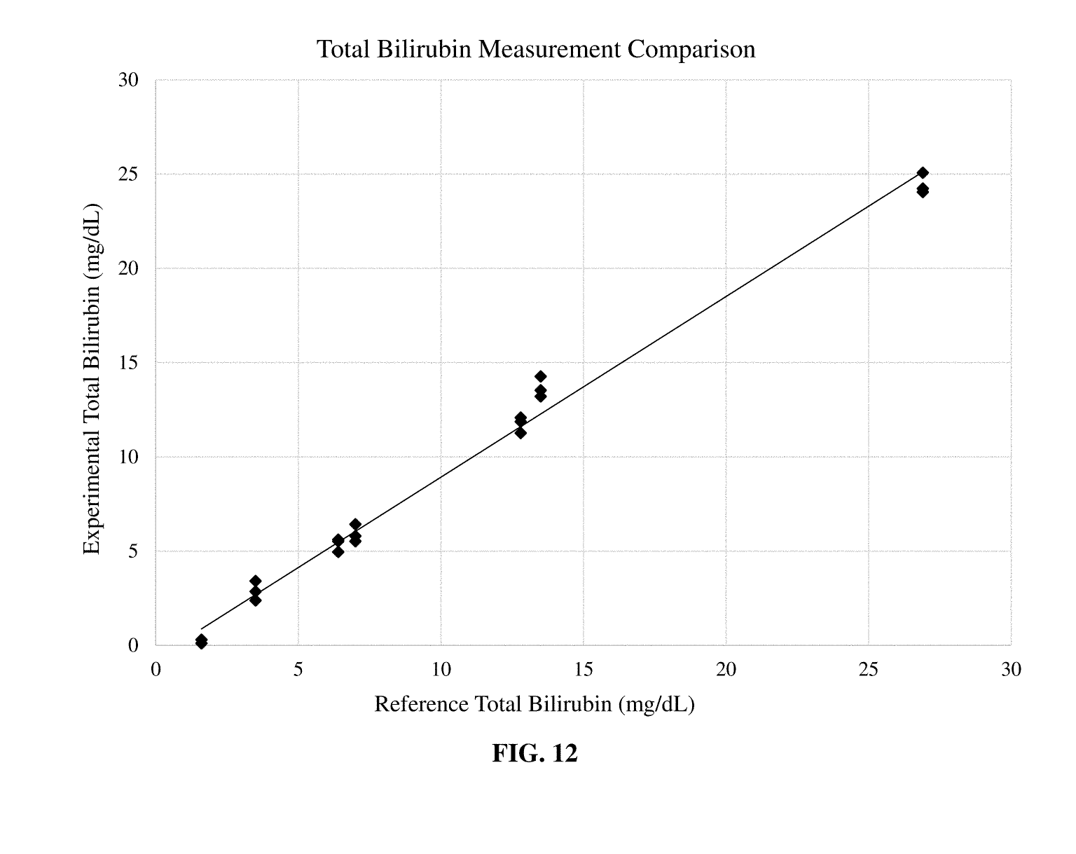

FIG. 12 depicts a graph showing a comparison between bilirubin measurements made using the optical sensor 100, 400, or other embodiments and assay described in the current invention compared to a reference bilirubin concentration measurement. The comparison demonstrates that the bilirubin concentration measurements made using the optical bilirubin sensor 100, 400, or other embodiments and assay described in the present invention are nearly identical to the reference measurements as demonstrated by the fact that the R² value for the trend line is 0.9901. The equation for the slope of the trend line is y=0.9582x−0.6561. In some embodiments, this equation may be used to convert the measured signal value calculated using Equation 1 to determine bilirubin concentrations in mg/dL.

The apparatus and methods disclosed herein for making an optical bilirubin measurement of a treated blood sample may be modified to allow for measurement with different geometries. For example, throughout this application, reference has been made to optically measuring for bilirubin concentration using a light source and detector positioned generally below the sensor wherein the light from the light source passes upward through the sample and is reflected back down to the detector. This is merely exemplary. One of skill in the art will understand, according to the principles herein disclosed, that the light source and detector could be positioned generally above the sensor. In some embodiments, the light source may be positioned on one side of the sensor and the detector could be positioned on the opposite side of the sensor such that the light emitted travels through a transparent portion of the lid or through a hole in the lid, through the sample, and through a transparent portion on the base of the sensor.

Accordingly, the embodiments and principles described above may be used to measure the bilirubin concentrations in a blood sample using a single sensor and analyzer.

### EXAMPLE 1

### Bilirubin Measurement

A sensor 400 and analyzer 200 incorporating the above-described principles for optically measuring bilirubin has been developed and tested yielding the following results. The analyzer was configured to calculate bilirubin using a preset bilirubin calibration curve.

The light intensity of a targeted and secondary wavelength reflected from the sensor 400 was measured before and after the sample was introduced. These wavelengths were approximately 480 nm and 525 nm respectively. The before and after intensity measurements were used to correct for stray and background absorbance. The adjusted 525 nm absorbance value was then subtracted from the targeted 480 nm wavelength. The concentration of bilirubin was determined using the calibration curve presented above. The same samples compared to reference values for. As shown in FIG. 12, there is an excellent correlation between the bilirubin concentrations determined using the principles herein disclosed and the reference value.

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the embodiments disclosed herein should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures may be combined, interchanged or excluded from other embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting.

What is claimed is:

1. A sensor for measuring bilirubin in a liquid sample, the sensor comprising:
a base layer comprising an optically transparent portion;
a one or more electrodes formed on the base layer;
a reflective layer, the reflective layer comprising:
at least a portion of a reservoir,
the reservoir configured to receive a sample to be analyzed.

2. The sensor of claim 1, wherein the sensor further comprises:
a filter layer disposed between at least a portion of a bottom surface of a lid and/or spacer layer and a top surface of a reflective layer.

3. The sensor of claim 1, wherein the sensor further comprises a spacer layer having a void extending through a thickness of the layer and wherein a portion of a bottom surface of the spacer layer may comprise a portion of the top surface of the reservoir;

4. The sensor of claim 2, wherein the sensor further comprises a spacer layer between the filter layer and the lid, the spacer layer having a void formed therein, the void extending through a thickness of the layer and wherein the size, shape, or thickness of the spacer layer determine the size, shape, or thickness of at least a portion of the filter layer.

5. The sensor of claim 1, wherein the sensor further comprises a lid, the lid having a void extending through a thickness of the lid, the lid having a bottom surface, and wherein at least a portion of the bottom surface of the lid forms at least a portion of the top surface of the spacer layer.

6. The sensor of claim 2, wherein the filter inhibits the passage of erythrocytes, white blood cells, and/or platelets.

7. The sensor of claim 2, wherein at least a portion of the filter may be compressed by the lid and/or spacer layer.

8. The sensor of claim 2, wherein a bilirubin binding material has been sputtered, printed, sprayed, air brushed, or otherwise deposited on at least a portion of the reflective layer.

9. The sensor of claim 1, further comprising at least one electrode disposed on a bottom surface of the reservoir and at least one electrical contact disposed on the substrate, and wherein the at least one electrode is in electrical communication with the at least one electrical contact.

10. The sensor of claim 2, further comprising at least one electrode disposed on a bottom surface of the reservoir and at least one electrical contact disposed on the base layer, and wherein the at least one electrode is in electrical communication with the at least one electrical contact.

11. A method for measuring bilirubin in a sample, the method comprising:
inserting a sensor into an analyzer, the sensor comprising a filter and the analyzer comprising a light source and a detector;
introducing the liquid sample to a sensor;
filtering the sample using the filter;
illuminating the liquid sample through a portion of the sensor using the light source;
measuring a reflectance of the liquid sample at one or more wavelengths using a detector in the analyzer; and
determining an amount of bilirubin based on the measured reflectances.

12. The method of claim 10, wherein the sample is whole blood and the filter inhibits the passage of erythrocytes.

13. The method of claim 10, wherein the sensor comprises a reflective layer, and reflectance is measured by measuring light reflected off the reflective layer.

14. The method of claim 10, further comprising taking a reference measurement in the sensor when no sample is present in the sensor.

15. The method of claim 10, wherein a reflectance is computed by comparing an intensity measured at the detector to a reference intensity.

16. The method of claim 14, wherein the reference intensity is obtained by inserting a reference sensor into the analyzer, illuminating reference sensor, and measuring an intensity of light received at the detector.

17. The method of claim 14, wherein internally reflected stray light is measured by detecting at the detector the intensity of light reflected off a light absorbing surface as the sensor is inserted into or withdrawn from the analyzer, the method further comprising subtracting the measured internally reflected stray light from the reference intensity and the measured intensity of the sample to obtain a result which adjusts for internally reflected stray light.

18. The method of claim 15, wherein determining the bilirubin concentration comprises: subtracting the secondary wavelength absorbance value from the adjusted target wavelength absorbance value to obtain a result that is corrected for hemoglobin and plasma interference, the method further comprising comparing the resulting value to a bilirubin calibration curve to determine the bilirubin concentration.

19. The method of claim 17, wherein the target wavelength is approximately 480 nm.

20. The method of claim 17, wherein the secondary wavelength is approximately 525 nm.
